(19) United States
(12) Patent Application Publication
(10) Pub. No.: US 2025/0262611 A1
(43) Pub. Date: Aug. 21, 2025

(54) COPPER-LOADED TWO-DIMENSIONAL SILICON CATALYST, PREPARATION METHOD THEREOF, AND ITS APPLICATION ON SOLAR-DRIVEN CATALYTIC CONVERSION OF POLYOLEFIN PLASTICS INTO HYDROCARBONS

(71) Applicant: ZHEJIANG UNIVERSITY, Hangzhou (CN)

(72) Inventors: WEI SUN, HANGZHOU (CN); CHUANWANG XING, HANGZHOU (CN); GEOFFREY ALAN OZIN, TORONTO (CA); DEREN YANG, HANGZHOU (CN)

(21) Appl. No.: 19/057,203

(22) Filed: Feb. 19, 2025

(30) Foreign Application Priority Data
Feb. 20, 2024 (CN) 2024101884272

(51) Int. Cl. B01J 23/72; B01J 21/06; B01J 35/39; B01J 37/02; B01J 37/08; C08J 11/16; C08J 11/20

(52) U.S. Cl. CPC B01J 23/72; B01J 21/06; B01J 35/39; B01J 37/0236; B01J 37/088; C08J 11/16; C08J 11/20; C08J 2323/06; C08J 2323/12

(57) ABSTRACT

A method for the solar-driven catalytic conversion of polyolefin plastics into hydrocarbon compounds, includes the following steps: The Cu nanoparticles is encapsulated by the two-dimensional silicon (2D Si) via a modified two-step impregnation-annealing method. 2D Si was dispersed in an ethanol solution containing copper salt. The mixture after removing the solvent is annealed at 250-300° C. for 4-6 hours under inert atmosphere. Then, the powder is washed with ethanol to remove unbound metal precursor. After drying in an oven, the obtained solid is annealed at 500-600° C. for 4-6 hours under inert atmosphere to obtain the copper-loaded 2D Si catalyst. Then, the catalyst, polyolefin plastics, and chlorinated tertiary alkane is dispersed in the chloroaluminate ionic liquid/chloroform solvent. Then, the reactor is under illumination with a light intensity of 4 suns, stimulating the photothermal effect of catalyst, which drives the depolymerization of the polyolefin plastic, resulting in the production of hydrocarbon products.

### TECHNICAL FIELD

The present invention relates to the field of conversion and utilization of plastic waste, specifically to a solar-driven catalytic conversion method of polyolefin plastics using copper-loaded two-dimensional silicon catalyst.

### BACKGROUND TECHNOLOGY

The worldwide plastics production has soared in the recent decades. It reached a staggering 390.7 million tons in 2021, with a substantial increase of 4% over the previous year (Nat. Nanotechnol. 2023, 18, 687). The gigantic plastics production causes severe financial losses and greenhouse gas emissions, as most plastics in use today are made from crude oil or gas. Based on current growth trends, 15% of the global carbon budget will be taken up by the carbon emissions derived from the plastic production in 2050. Globally, only 9% of plastic waste is recycled, while most of it is destined for landfills, incineration or leaked into the nature as waste, resulting in serious environmental and health disasters (Adv. Mater. 2021, e2100843). It is imperative to design approaches for plastic waste management.

Among the plastic recycling approaches, chemical upcycling is a promising technique that can transform waste into value-added products, allowing synchronous environmental remediation and chemical production. However, its industrial practice is challenging since the economic effectiveness of products cannot recover the costs of the upcycling process. In particular, depolymerization of polyolefins, which account for >50% of global plastic materials and waste, is constrained by the extremely inert C—C bonds. Thermocatalytic hydrogenolysis techniques for polyolefins have demonstrated some progress in recent years. However, the harsh operating conditions, including high temperatures (˜300° C.), high hydrogen pressure and the associated special reacting equipment, and massive energy input, significantly increase the cost of polyolefin upcycling (J. Am. Chem. Soc. 2022, 144, 14269; Nat. Catal. 2018, 2, 46). Utilizing inexpensive, green solar energy to convert plastic waste into high-value hydrocarbons is an effective way to alleviate the global plastic waste crisis.

Traditional photocatalytic plastic conversion uses photo-generated carriers from the photocatalyst to drive the redox degradation of plastics. Unfortunately, although these photochemical recycling can proceed under mild conditions, their extremely low yield rates (typically at the level of μmol g_{cat}⁻¹ h⁻¹) suppress the practical application. Meanwhile, as an emerging plastic upcycling approach, photothermal catalysis outstands with the features of clean energy input and excellent conversion efficiency.

Very recently, the literature “Photothermal recycling of waste polyolefin plastics into liquid fuels with high selectivity under solvent-free conditions” (Nat. Commun. 2023, 14, 4242) reported a photothermal system for the hydrogenolysis of polyolefin, reaching almost complete transformation of the plastic waste in a short period (3 hours). Nevertheless, the high light intensity of tens of Suns is required to obtain a high operating temperature, resulting in a small irradiated area due to the employment of solar concentrator.

Recent reports have suggested that polar environments can stabilize ionic intermediates in plastic cracking reactions, thereby decreasing reaction temperature. For instance, the literature “Low-temperature upcycling of polyolefins into liquid alkanes via tandem cracking-alkylation” (Science 2023, 379, 807) constructed a highly polar reaction environment using an ionic liquid solvent. With (isopentane) iC₅ as the coreactant, the low-density polyethylene (LDPE) was completely converted into isoalkanes under a low temperature of 70° C. However, this strategy consumes a stoichiometrically equivalent number of iC₅ chemical to recycle the cheap plastic waste, which may restrict its economy of scale.

Based on the aforementioned results, there are still enormous opportunities in developing novel photothermal catalysts and systems for promoting the feasibility and scalability of green, mild, and efficient upcycling of polyolefins.

### SUMMARY OF THE INVENTION

In order to solve the problems existing in the prior art, the present invention provides a low-temperature and coreactant-free solar thermal system to catalytically upcycle polyolefins into high-value hydrocarbons. This mild process is demonstrated to offer energy, economic and environmental benefits. The enabling catalyst is an ionic liquid suspension of a two-dimensional multilayer silicon material with copper nanoparticles encapsulated between the layers (denoted as Cu/2D Si). The catalyst is a broad band absorber of sunlight in a highly polar reaction environment designed to efficiently harvest solar photons while lowering the energy of ionic transition states. This strategy enables rapid and total conversion of polyethylene to C₃ to C₂₆ hydrocarbons at 55° C., a notably mild temperature created photothermally at just mild light intensity.

The present invention provides a method to prepare copper-loaded two-dimensional silicon catalysts as the following steps:
(1) two-dimensional silicon is dispersed in an ethanol solution containing copper salts, and the solvent of the mixture is then removed by evaporation. The solid thus obtained is then annealed at 250-300° C. for 4-6 hours under nitrogen atmosphere, obtaining a solid powder;
(2) the solid powder is washed with ethanol and centrifugation-collected three times to remove unbound metal precursor;
(3) after drying in an oven, the resulting solid obtained from step (2) is annealed at 500-600° C. or 4-6 h in a nitrogen flow to obtain the Cu/2D Si catalyst.

The present invention describes a method for preparing a Cu/2D Si catalyst. The process involves first dispersing 2D Si in an ethanol solution containing copper salts. After removing the solvent, the solid mixture is annealed at 250-300° C., and the resulting powder is washed to remove unbound metal precursor. Next, the obtained product is subjected to a second annealing process at 500-600° C. During the process, the silicon nanosheets in situ reduced Cu²⁺ into Cu⁰, resulting in Cu nanoparticles embedded in the stacked 2D Si structure. The Cu/2D Si catalyst takes the merits of the outstanding dehydrogenation activity of metallic Cu nanoparticles (NPs) and excellent solar light absorption of silicon nanosheets (NSs), enabling solar-driven polyolefin conversion.

Preferably, the preparation method comprises the following steps:
(1) Two-dimensional silicon was dispersed in an ethanol or water solution containing copper salts. The mixture was sonicated for 10-30 min and then maintained at 80° C. to remove solvent. The obtained powder was annealed at 250-300° C. with a ramping rate of 5° C. min⁻¹ for 4-6 hours under nitrogen atmosphere, allowing the bonding of metal precursors to the 2D Si support.
(2) The solid powder is washed with ethanol and centrifugation-collected three times to remove unbound metal precursor.
(3) After drying in an oven, the resulting solid was annealed at 500-600° C. with a ramping rate of 2° C. min⁻¹ for 4-6 hours in a nitrogen flow to obtain the copper-loaded two-dimensional silicon catalyst (Cu/2D Si catalyst).

The Cu/2D Si catalyst takes the merits of the outstanding dehydrogenation activity of metallic Cu nanoparticles (NPs) and excellent solar light absorption of silicon nanosheets (NSs).

The two-dimensional silicon material can be prepared by conventional methods such as exfoliating calcium silicide with hydrochloric acid and stripping silicon powder using ultrasonic method.

Preferably, the copper salt includes one or more of copper chloride, copper sulfate or copper nitrate.

Preferably, in step 1, the mass ratio of silicon to copper is 1:0.03-0.2. If the copper loading is too low, the catalytic performance of the material decreases. If the copper loading is too large, the loaded copper particles are prone to aggregation, which also reduces the catalytic performance.

Preferably, in step 2, ethanol and water are used to wash the solid powder obtained in step 1 to remove the copper salt that is not bonded to the two-dimensional silicon material.

The present invention also introduces copper-loaded two-dimensional silicon catalysts prepared by the above-mentioned method. The two components of Cu/2D Si can synergistically capture the whole solar spectrum, stimulating the local photothermal heating to drive the complete conversion of polyolefins into high-yield, high-value hydrocarbons.

Preferably, the copper content in the copper-loaded two-dimensional silicon catalyst should range from 3 to 20 wt %. The catalytic performance of the Cu/2D Si catalyst is at its best when the copper content is 6 wt %.

The present invention also provides the application of a copper-loaded two-dimensional silicon catalyst for the solar-driven catalytic conversion of polyolefin plastics into hydrocarbon compounds. The method utilizes a copper-loaded two-dimensional silicon material as the catalyst, a chloroaluminate ionic liquid/trichloromethane mixture as the solvent, and a chlorinated tertiary alkane as the initiator. Under light exposure, polyolefin plastics can be completely converted into small hydrocarbon molecules. The process significantly lowers the reaction temperature for polyolefin cracking to 55° C., achieving 100% conversion. This advancement is highly significant for the environmentally friendly recycling of plastic waste.

Preferably, the application method is: under an inert atmosphere, the polyolefin plastic is mixed with a copper-loaded two-dimensional silicon catalyst, a chloroaluminate ionic liquid/chloroform solvent, and a chlorinated tertiary alkane initiator using magnetic stirrer. Then the reaction system was irradiated with light to drive the depolymerization of the polyolefin, resulting in the production of small molecule hydrocarbons.

The chloroform solvent in the present invention can effectively swell polyolefin plastics, facilitating contact between the catalyst and the plastic substrate. The chloroaluminate ionic liquid establish a high-polarity reaction environment, which can stabilize highly polar carbon cation intermediates in polyolefin cracking reactions. The chlorinated tertiary alkane acted as an initiator to offer an initial concentration of carbocations. The two components of Cu/2D Si catalyst can synergistically capture the whole solar spectrum, stimulating the local photothermal heating to drive the upcycling.

Preferably, the chloroaluminate ionic liquid/chloroform solvent is prepared by mixing N-butyl pyridinium chloride ([C₄Py]Cl), anhydrous AlCl₃, and chloroform in a flask. Note that the preparation process was performed in a nitrogen atmosphere and cooled with an ice-water bath.

Preferably, the amount of the [C₄Py]Cl, anhydrous AlCl₃ and chloroform is 6 mmol:12 mmol:3-6 mL. When the molar ratio of [C₄Py]Cl to anhydrous AlCl₃ is 1:2, the sample exhibits optimal performance.

Preferably, the stirring speed is 600-1200 rpm, and the stirring duration is 5-20 minutes.

Preferably, the chlorinated tertiary alkane includes one or more of chlorinated tertiary butylene, chlorinated tertiary pentane or chlorinated tertiary hexane.

Preferably, the amounts are as follows: polyolefin plastic (200-1000 mg), Cu/2D Si catalyst (10-50 mg), ionic liquid/chloroform solvent (3 mL), and chlorinated tertiary alkane (10-50 μL).

Preferably, the light sources for the irradiation reaction system include sunlight, focused sunlight, mercury lamp, halogen tungsten lamp, LED or a Xenon lamp.

Preferably, polyolefin plastic substrates include low-density polyethylene, high-density polyethylene, polypropylene, polyvinyl chloride, and combinations thereof.

Preferably, polyolefin plastic substrates include post-consumer low-density polyethylene, high-density polyethylene, polypropylene, polyvinyl chloride products, and combinations thereof.

Preferably, the irradiation intensity of the light source is 100-500 mW/cm², and the irradiation time is 5-12 hours.

The upcycling can proceed under pure light illumination with a low intensity of 4 Suns, at an incredibly lower temperature (55° C.) compared with conventional thermal techniques. The polyethylene can be completely transformed into distinct and separable distributions of alkanes (C₃-C₇) and cyclic hydrocarbons (C₈-C₂₆) within hours, with a total yield of 91%.

Compared to the prior technique, the present invention offers at least the following advantages:

(1) The catalyst of the present invention. leverages the outstanding dehydrogenation activity of copper nanoparticles, combined with the superior light absorption and photothermal conversion properties of silicon nanosheets, to achieve efficient light-driven polyolefin conversion. The catalyst features facile preparation process, low cost, and abundant raw materials.

(2) The present invention enables the conversion of polyolefins driven by clean, pollution-free solar energy. The mild reaction conditions make this method suitable for large-scale applications. Additionally, the process achieves complete conversion of polyolefin plastics, yielding high-value hydrocarbon compounds in high quantities. The present invention offers innovative insights for the green and value-added recycling of plastic waste.

(3) The present invention eliminates the need for high temperatures, high pressures, hydrogen, and specialized reaction equipment typically required in thermal catalytic conversion processes. Meanwhile, it overcomes the low production rates associated with conventional photocatalytic conversion methods. Additionally, the present invention requires significantly lower light intensity compared to recent photothermal catalytic models and supports a larger reaction scale.

### BRIEF DESCRIPTION OF THE DRAWINGS

The present invention is further described with drawings and examples below:

FIG. 1: Morphology of Cu/2D Si catalyst prepared in Example 1, as observed under a transmission electron microscope.

FIG. 2: Conversion rate of polyolefin plastics and yield of hydrocarbons using the catalyst from Example 1 at a light intensity of 400 mW/cm².

### DETAILED EMBODIMENTS

### Example 1

### Preparation of 2D Si-Encapsulated Copper Nanoparticle (Cu/2D Si)

400 mL of hydrochloric acid was added into a jacketed flask and purged with nitrogen for 30 min to eliminate oxygen. The reaction was kept at between −20° C. and −25° C. by circulating liquid (ethylene glycol/water) in the interlayer of the flask. Then, 4.00 g of calcium silicide powder was added to the hydrochloric acid and stirred under nitrogen atmosphere for 7 days. The remaining solid (stacked 2D Si) was collected by centrifugation and washed thoroughly with ethanol and dried at 60° C. under vacuum.

The Cu nanoparticles were encapsulated by the 2D Si via a modified two-step impregnation-annealing method. 0.50 g of 2D Si was dispersed in an ethanol solution containing 0.86 g of CuCl₂·2H₂O (6 wt % metal feeding). The mixture was sonicated for 30 min and then maintained at 80° C. to remove solvent. The obtained powder was annealed at 300° C. with a ramping rate of 5° C. min⁻¹ for 5 hours under nitrogen atmosphere, allowing the bonding of metal precursors to the 2D Si support. Then, the powder was washed with 30 ml ethanol and centrifugation-collected three times to remove unbound metal precursor. Finally, after drying in an oven, the obtained solid was annealed at 550° C. with a ramping rate of 2° C. min⁻¹ for 5 hours in a nitrogen flow.

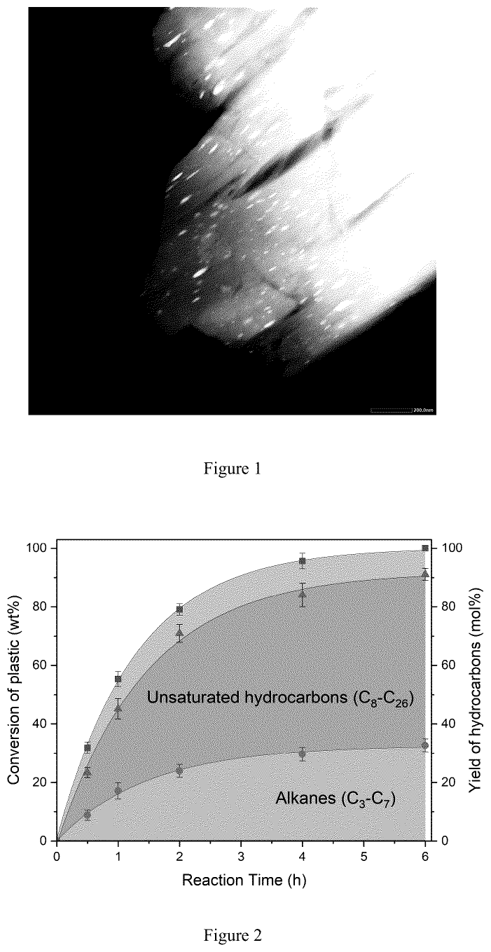

FIG. 1 shows the morphology of the Cu/2D Si catalyst prepared in this example, as observed under a transmission electron microscope. The image reveals that the two-dimensional silicon exists in the form of nanosheet stacking. The white bright spots represents Cu nanoparticles confined within the stacked nanosheets of silicon.

### (2) Preparation of Chloroaluminate Ionic Liquid/Chloroform Solvent

In a typical preparation of highly polar reaction solvent, 6 mmol of N-butyl pyridinium chloride ([C₄Py]Cl), 12 mmol of anhydrous AlCl₃, and 3 mL of trichloromethane were mixed in a quartz flask. Note that the preparation process was performed in a nitrogen atmosphere and cooled with an ice-water bath. A homogeneous light-grey liquid was obtained under magnetic stirring.

### (3) Solar-Driven Polyolefin Conversion

10 mg of Cu/2D Si, 200 mg of LDPE, and 10 μL of tert-butyl chloride (TBC) were dispersed in the solvent. The TBC acted as an initiator to offer an initial concentration of carbocations. Then, the reactor was under a simulated sunlight illumination from a Xe lamp equipped with a AM 1.5 optical filter with a light intensity of 4 suns, stimulating the photothermal effect of Cu/2D Si to reach the specific reaction temperature. The temperature was kept for a 6-hours reaction time.

FIG. 2 illustrates the conversion rate of polyolefin plastics and the yield of hydrocarbons in the reaction. The cube data points represent the polyolefin conversion rate, which reaches 100% after 6 hours. The dot data points indicate the alkane yield, while the difference between the triangle and dot data points represents the yield of unsaturated hydrocarbons. It can be observed that the combined yield of alkanes and unsaturated hydrocarbons approaches approximately 90% within 6 hours.

### Example 2

In this example, the key difference from Example 1 is that the light source used is natural sunlight focused by a Fresnel lens, with the light power maintained at 400-500 mW/cm². During the solar-driven polyolefin conversion reaction, after 6 hours of illumination, the plastic conversion rate reaches 100%, and the hydrocarbon yield achieves 86%.

### Example 3

In this example, the polyolefin powder from Example 2 is replaced with post-consumer low-density polyethylene plastic bags. The light power is controlled at 400-500 mW/cm². During the solar-driven conversion reaction of the polyolefin plastic bags, after 6 hours of illumination, the plastic conversion rate reaches 100%, and the hydrocarbon yield achieves 91%.

### Example 4

In this example, the polyolefin powder from Example 2 is replaced with chopped commercial high-density polyethylene plastic bottles. The light power is maintained at 400-500 mW/cm². During the solar-driven conversion reaction of the high-density polyolefin plastic bottles, after 12 hours of illumination, the plastic conversion rate reaches 100%, and the hydrocarbon yield is 80%.

### Example 5

In this example, the polyolefin powder from Example 2 is replaced with post-consumer low-density polyethylene, high-density polyethylene, polypropylene, polyvinyl chloride, and combinations thereof. The light power is maintained at 400-500 mW/cm². During the solar-driven conversion reaction of the mixed feedstocks, after 12 hours of illumination, the plastic conversion rate reaches 100%, and the hydrocarbon yield is 73%.

1. A preparation method of copper-loaded two-dimensional silicon (Cu/2D Si) catalyst comprising the following steps:
(1) dispersing two-dimensional silicon in an ethanol or water solution containing copper salts, then removing the ethanol or water solvent of the mixture by evaporation, obtaining a solid then annealing the solid at 250-300° C. for 4-6 hours under nitrogen atmosphere, obtaining a solid powder;
(2) washing the solid powder with ethanol and centrifugation-collected the solid power three times to remove unbound metal precursor;
(3) after drying in an oven, annealing a resulting solid obtained from step (2) at 500-600° C. for 4-6 hours in a nitrogen flow to obtain the Cu/2D Si catalyst.

2. The preparation method according to claim 1, wherein, a molar ratio of the silicon element in the two-dimensional silicon to the copper element in the copper salt is 1:0.03-0.2.

3. The copper-loaded two-dimensional silicon catalyst according to claim 1, wherein, the content of copper in the catalyst is 3-20 wt %.

4. A copper-loaded two-dimensional silicon catalyst prepared by the method according to claim 1.

5. A copper-loaded two-dimensional silicon catalyst prepared by the method according to claim 2.

6. A copper-loaded two-dimensional silicon catalyst prepared by the method according to claim 3.

7. A method for solar-driven catalytic conversion of polyolefin plastics into hydrocarbon compounds, comprising the step of utilizing the Cu/2D Si catalyst according to claim 4.

8. The method according to claim 7, wherein, in an inert atmosphere, polyolefin plastics, Cu/2D Si catalyst, chloroaluminate ionic liquids/trichloromethane solvents and chlorinated tertiary alkanes are mixed and stirred, and the reaction system is irradiated with light to drive the depolymerization of the polyolefin plastics to obtain hydrocarbon compounds.

9. The method according to claim 8, wherein, a preparation method of the chloroaluminate ionic liquid/chloroform solvent is as follows: under an inert atmosphere, N-butyl pyridinium chloride, anhydrous aluminum chloride and chloroform are mixed; an amount of N-butyl pyridinium chloride, anhydrous aluminum chloride and chloroform is 6 mmol:12 mmol:3˜6 mL.

10. The method according to claim 8, wherein, the amounts of the polyolefin plastic, Cu/2D Si catalyst, chloroaluminate ionic liquid/chloroform solvent and chlorinated tertiary alkane are 200˜1000 mg: 10˜50 mg: 3 mL: 10˜50 μL, respectively.

11. The method according to claim 8, wherein, the light source used in the reaction system includes sunlight, focused sunlight, mercury lamp, halogen tungsten lamp, LED or a Xenon lamp.

12. The method according to claim 11, wherein, the irradiation intensity of the light source is 100-500 mW/cm², and the irradiation time is 5-12 hours.

13. The method according to claim 8, wherein, the plastic substrates comprise low-density polyethylene, high-density polyethylene, polypropylene, polyvinyl chloride, and combinations thereof.

14. The method according to claim 8, wherein, the plastic substrates include post-consumer low-density polyethylene, high-density polyethylene, polypropylene, polyvinyl chloride products, and combinations thereof.

15. The method according to claim 9, wherein, the amounts of the polyolefin plastic, Cu/2D Si catalyst, chloroaluminate ionic liquid/chloroform solvent and chlorinated tertiary alkane are 200˜1000 mg: 10˜50 mg: 3 mL: 10˜50 μL, respectively.

16. The method according to claim 9, wherein, the light source used in the reaction system includes sunlight, focused sunlight, mercury lamp, halogen tungsten lamp, LED or a Xenon lamp.

17. The method according to claim 16, wherein, the irradiation intensity of the light source is 100-500 mW/cm², and the irradiation time is 5-12 hours.
